(19) United States
(12) Patent Application Publication
(10) Pub. No.: US 2026/0231961 A1
(43) Pub. Date: Aug. 13, 2026

(54) RUBISCO IN CAKE

(71) Applicant: MAURI TECHNOLOGY B.V., Etten-Leur (NL)

(72) Inventors: Neil Owen CARR, Etten-Leur (NL); Maarten VAN OORT, Etten-Leur (NL)

(73) Assignee: MAURI TECHNOLOGY B.V., Etten-Leur (NL)

(21) Appl. No.: 19/149,731

(22) PCT Filed: Jan. 31, 2024
(86) PCT No.: PCT/NL2024/050046

§ 371 Date: Jul. 21, 2025

(30) Foreign Application Priority Data
Feb. 1, 2023 (EP) 23154521.1

(51) Int. Cl. A21D 13/064; A21D 2/18; A21D 2/26; A21D 13/80; A23L 15/00

(52) U.S. Cl. CPC A21D 13/064; A21D 2/186; A21D 2/262; A21D 2/264; A21D 13/80; A23L 15/35

(57) ABSTRACT

The invention relates to a dough, batter or instant food mixture, comprising a non-denatured ribulose-1,5-bisphosphate carboxylase-oxygenase (RuBisCo) preparation, a starch, a sweetener and water and to a heated food product, preferably a baked or a fried food product, more preferably a cake, comprising a heated dough or batter according to the invention.

The invention relates to a batter, a dough, an instant food mixture, a heated food product, a method for preparing a heated food product and a use of a non-denatured RuBisCo preparation as an egg substitute or a binder in a food product.

Cake is a traditional sweet bakery product of which numerous varieties are known, such as sponge cakes, pound cakes, chiffon cakes, genoise cakes, cupcakes and muffins.

Typical cake ingredients include (wheat) flour, eggs and sugar. Further ingredients, such as baking powder, emulsifiers, thickeners, humectants, flavoring, water, milk or fat, such as oil, butter or margarine may also be optionally added.

In the preparation of cakes, eggs typically play a key role in providing sufficient structure to the crumb of the cake. Upon heating of the eggs, the proteinaceous fraction present in egg white coagulates (in addition to gelatinization of starch), thereby forming a solid matrix, along with the gelatinized starch from the flour and similar, to provide structure to the cake. Furthermore, gas bubbles are entrapped in the crumb, to provide a light and voluminous crumb texture that is characteristic for cakes.

There is a global trend to reduce the daily intake of eggs, for various reasons, for instance due to concerns relating to animal welfare, global warming and the risk of disease associated with keeping birds, such as avian flu or with storing animal-derived products, including eggs, such as Salmonella contamination. For this reason, there is an increasing trend in consumers to remove animal-derived products, including eggs from their diet.

Moreover, it is estimated that about 2% of children are allergic to eggs, and therefore need to remove eggs from their diet.

Accordingly, there is a need for food products, in particular cakes, in which the presence of eggs is not required.

It has been found that simply removing eggs from cake, typically does not provide a satisfactory structure and texture of the cake, because such a cake lacks the support normally provided by the egg white proteins present in eggs. Accordingly, there is a need for an egg replacer in food products, and in particular in sweet bakery products such as cakes.

There are several products on the market that aim to replace eggs in bakery products, including cake. Examples include Orgran No Egg™ Egg Replacer and Follow Your Heart VeganEgg™. However, cakes based on these egg replacers were either too fragile to be removed from their baking tins, or exhibited unsatisfactory texture, in particular low crumb hardness and low specific cake volume compared to a reference cake based on egg white.

Accordingly, there is still a need for an egg-replacer in food products, that allows replacement of eggs in food products, in particular in cake, whilst maintaining satisfactory cake properties, such as specific cake volume and cake texture. It is in particular an object of the present invention to prepare a food product, in particular a cake, that has comparable cake properties, in particular comparable specific volume and/or crumb hardness, compared to a comparable reference cake comprising egg white.

The inventors surprisingly realized that with a ribulose-1,5-bisphosphate carboxylase-oxygenase (RuBisCo) preparation a cake may be prepared that has satisfactory properties such as a satisfactory specific cake volume or cake texture, without requiring to use eggs.

Accordingly, the invention relates to a dough, batter or instant food mixture, comprising a non-denatured ribulose-1,5-bisphosphate carboxylase-oxygenase (RuBisCo) preparation, a starch, sweetener and water.

The invention further relates to a heated food product, preferably a baked or a fried food product, comprising and a RuBisCo preparation, a starch, sweetener, in particular made from a dough according to the invention or comprising a heated dough or batter according to the invention.

The invention further relates to a method for preparing a heated food product, preferably a heated food product according to the invention, comprising mixing a starch, a sweetener, a non-denatured RuBisCo preparation and water to form a batter or a dough, preferably a batter of dough according to the invention; or mixing an instant food mixture, comprising a starch, a sweetener, a non-denatured RuBisCo preparation, preferably an instant food mixture according to the invention, with water to form a batter or a dough, preferably a batter or dough according to the invention; heating said batter or dough to a temperature above the denaturing temperature of said non-denatured RuBisCo to obtain the heated food product.

The invention further relates to the use of a non-denatured RuBisCo preparation as an egg substitute or egg white substitute (in the preparation) of a food product, in particular a heated food product, or a dough or batter, preferably a dough, batter of food product according to the invention.

The invention further relates to the use of a non-denatured RuBisCo preparation as a binder in a food product, preferably a heated food product, or a dough or batter, preferably a dough, batter of food product according to the invention.

### FIGURES

FIG. 1: Cross-sectional images of cakes from cakes of Table 3; A: Egg-free with 1 wt. eq. of RuBisCo protein; B: Egg-Free with 1.5 wt. equivalent of RuBisCo; C: Egg Free with 1 wt. equivalent of RuBisCo and with lecithin; D: Egg free with 1 wt. equivalent of RuBisCo and with lecithin and with adjusted leavening salts; E: reference cake with whole liquid egg; F: reference cake with liquid egg yolk and dried egg white; G: reference cake with dried egg white only; H: reference cake without egg and without RuBisCo.

FIG. 2: Plot of crumb hardness against specific volume for cakes of Table 3.

FIG. 3: Cross-sectional images of cakes from cakes of Table 6; A: Egg-free with 1 wt. eq. of RuBisCo protein; B: Egg-Free with 1.5 wt. equivalent of RuBisCo; C: Egg Free with 1 wt. equivalent of RuBisCo and with lecithin; D: Egg free with 1 wt. equivalent of RuBisCo and with lecithin and with adjusted leavening salts; E: Egg free with 1 wt. equivalent of RuBisCo and with lecithin, adjusted leavening salts and with no added starch; F: reference cake with whole egg; G: reference cake with liquid yolk and dried egg white; H: reference cake with dried egg white only; I: reference cake without egg and without RuBisCo;

FIG. 4: Plot of crumb hardness against specific volume for crème cakes of Table 6.

FIG. 5: Images of cake doughnuts of Table 9; A: Left to right Trial 1, 3, 4 and 5; B: Trial 2; B: Trial 2.

FIG. 6: Images of cakes from Table 12 that could be removed from baking tins; F: reference cake with whole egg; H: reference cake with dried egg white; M: Egg-free cake with FollowYourHeart; A: Egg-free with 1 wt. eq. of RuBisCo protein.

### DETAILED DESCRIPTION

The term “or” as used herein is defined as “and/or” unless specified otherwise.

The term “a” or “an” as used herein is defined as “at least one” unless specified otherwise.

The term “substantial(ly)” or “essential(ly)” is generally used herein to indicate that it has the general character or function of that which is specified. When referring to a quantifiable feature, these terms are in particular used to indicate that it is for at least 75%, more in particular at least 90%, even more in particular at least 95% of the indicated feature.

When referring to a noun in the singular, the plural is meant to be included, unless it follows from the context that it should refer to the singular only.

The terms “protein” and “polypeptide” are used to refer to at least one chain of amino acids arranged in a specific order (determined by the coding sequence in a polynucleotide encoding the polypeptide). Typically, the chain comprises at least 10 amino acids or more, preferably at least 15 amino acids or more, such as 20 amino acids or more. There is no maximum number of amino acids that may be present in a protein, although generally speaking, a protein has at most 40.000 amino acid residues, such as 35.000 amino acid residues. On average, a protein typically comprises about 100 and about 1500 amino acid residues.

A protein may be in any shape or form. The primary structure of a protein refers to the linear sequence of amino acid residues. A secondary structure of a protein refers to a regular substructure of amino acid residues, wherein the amino acid residues of a protein typically engage in intramolecular interactions with the backbone to form a two-dimensional structure, such as an α-helix or a β-strand, also referred to as β-sheet. Generally, a protein in a tertiary structure, is involved in a plurality of intramolecular polar interactions, such as ionic interactions or hydrogen bonding interactions and intramolecular nonpolar interactions, such as van der Waals interactions or n-n stacking interactions and/or reversible covalent bonds such as disulphide bonds. Typically, a protein in its native form assumes a specific tertiary structure. Said tertiary structure is typically reasonably fixed in a stable environment, such as in the absence of a change in temperature or change of medium wherein the protein is present, to provide a required biological function.

The term “protein preparation” as used herein refers to an isolate of one or more proteins that has been at least substantially isolated from non-protein components that were present in a natural product in which it naturally occurs, typically a plant or plant-part thereof.

With the term “denatured” as used herein is meant a protein that has at least substantially lost the tertiary structure as found in its native (i.e. non-denatured) state. A protein can typically become denatured by applying an external stimulus to promote substantial unfolding of a protein, typically by disrupting intramolecular interactions that stabilize the tertiary structure of a protein. Examples of external stimuli that may promote unfolding of a protein include high salt organic solvents, pH or heat.

A protein in its denatured state typically forms an aggregate, wherein the hydrophobic portions of a protein interact with hydrophobic portions of other denatured proteins, and/or wherein the hydrophilic portions are typically oriented outwards to interact with water molecules or other polar groups.

With the term “batter” as used herein is generally meant a mixture comprising at least a starch (e.g. starch present in cereal flour), sweetener and water that has a viscosity such that it is pourable at 25° C. and atmospheric pressure. Typically, a batter is not able to hold its shape at 25° C. and atmospheric pressure, in absence of a tool holding the batter in place, such as a container. Upon heating of a batter (typically above a temperature of at least 40° C.), a solid food product is generally obtained that is no longer pourable.

With the term “dough” as used herein, is typically meant a mixture comprising at least a starch, sweetener and water, the dough having a viscosity that exceeds the viscosity of a batter. A dough is typically kneadable using a suitable tool such as hands or a mechanical mixer and is typically not pourable. A dough is typically able to substantially hold its shape in absence of a tool holding it in place, e.g. a container. Upon heating of a dough (typically above a temperature of at least 60° C.), a solid food product is generally obtained that is no longer kneadable.

In the context of the present application, the term “egg” as used herein refers to an avian egg, such as eggs from chicken (hen), turkey, goose, duck, guinea fowl, pheasant, quail, ostrich and emu. As the skilled person will understand, an egg typically comprises an ‘egg white’ (which, in uncooked form is a translucent fluid), that is rich in protein, and an ‘egg yolk’ (which, in uncooked form, is a yellow fluid that is separated from said egg white by a barrier layer), that is typically rich in fat. Included in the term ‘egg’ are “whole eggs”, i.e. including both the egg white and egg yolk, and isolated fractions thereof, such as egg white, egg yolk, the solid matter of egg (which can e.g. be obtained by freeze-drying or spray-drying a whole egg), the protein fraction or lipid fraction of a whole egg.

With the term “cake” as used herein is meant a sweet, non-fermented bakery product prepared from heating a dough or batter. A cake has a “void volume”—the volume of the cake that is gaseous—of between about 50 vol. % and about 90 vol. % as determinable with X-ray microCT using the method described in Example 1. The void volume is due to the presence of gas bubbles entrapped in the heated dough or batter. Said gas bubbles may be formed by mixing air into the batter or the dough, and/or by including a leavening agent such as sodium bicarbonate, in the batter or dough.

### Batter, Dough, Instant Food Mixture

The inventors realized that the structure-providing properties of egg white to food products may be successfully mimicked by non-denatured RuBisCo. This allows for the preparation of a food product, in particular a cake, that has satisfactory properties in terms of specific (cake) volume, organoleptic properties, (cake) texture and the like, without requiring to include egg or a fraction thereof in the food product, such as a cake.

Exclusion of egg from food products is an advantage, because this makes the food product suitable for consumers that do not consume egg, for example due to an allergy or because they have excluded animal-derived products from their diet (also known in the art as a vegan diet). Furthermore, the food product advantageously does not depend on the price of eggs, which may fluctuate due to various reasons and may contribute to an overall reduction of poultry required in the agricultural industry to produce eggs.

Accordingly, the invention relates to a dough, batter or instant food mixture, comprising a non-denatured RuBisCo preparation, a starch, sweetener and water.

Preferably, the invention relates to a batter, comprising a non-denatured RuBisCo preparation, a starch, a sweetener and water.

### RuBisCo Preparation

RuBisCo constitutes a class of enzymes that catalyses the carboxylation reaction of the sugar ribulose-1,5-bisphosphate. RuBisCo is the most abundant protein on earth, and can comprise up to 50% of total soluble protein found in leaf tissue of plants (Tabita et al., Microbiology and molecular biology reviews, 2007, 71 (4): 576-599).

Four subtypes of RuBisCo have been presently identified, referred to in the art as Form I-IV, wherein Form I is typically found in plants or algae. Preferably, RuBisCO preparation comprises RuBisCo Form I. RuBisCo Form I typically comprises a protein structure of the form (L₂)₄(S₄)₂, wherein L is a large subunit of about 55.000 Da and wherein S is a small subunit of about 15.000 Da. Typically, the total molecular weight of a RuBisCo protein is about 550.000 Da.

A non-denatured RuBisCo preparation may comprise a non-denatured RuBisCo from any vegetable source. Preferably, said non-denatured RuBisCo preparation comprises a non-denatured RuBisCo from the stem, root, fruit, flower or leaf of a plant, more preferably from a leaf of a plant.

Preferably, non-denatured RuBisCo preparation comprises a non-denatured RuBisCo from duckweed, algae, beetroot, spinach, spinach beet, kale, beet, sugar beet, chard, sea beet, Mangel αeet, carrot, sugar beet, corn, pulses, alfalfa, soy or tobacco, more preferably from a leaf thereof.

Most preferably, said non-denatured RuBisCo preparation comprises a RuBisCo protein from duckweed. Duckweed advantageously has the ability to double biomass in about 16 to 48 hours depending on the conditions, and typically has a protein content of about 30-35 wt. % based on the total dry mass. These properties of duckweed make it an attractive source of non-denatured RuBisCo.

Preferably, said RuBisCo preparation comprises a RuBisCo protein from a plant of the Family Araceae, more preferably from a plant of the subfamily Lemnoideae, in particular from a plant of the genus Lemna.

Examples of species of the genus Lemna include Lemna aequinoctialis, Lemna perpusilla, Lemna tenera, Lemna disperma, Lemna ecuadoriensis, Lemna gibba, Lemna japonica, Lemna minor, Lemna obscura, Lemna trisulca, Lemna turionifera, Lemna minuta, Lemna valdiviana and Lemna yungensis.

Preferably, said non-denatured RuBisCo preparation comprises a non-denatured RuBisCo from the leaves of a plant, preferably from the leaves, of L. minor.

Preferably, said RuBisCo is isolated from a plant or plant part thereof. Said RuBisCo may be isolated from said plant or plant part using any suitable method known in the art allowing isolation of RuBisCo from a natural product in its native, non-denatured, state, thereby obtaining a non-denatured RuBisCo preparation.

Preferably, said RuBisCo preparation is obtainable with a process for making a purified protein preparation from a plant material, comprising
a) Providing the plant material in a buffer solution comprising a reducing agent;
b) Lysing the plant material;
c) Separating the lysed plant material into a solid phase and a liquid phase, wherein the liquid phase contains soluble protein and chlorophyll;
d) Coagulating the chlorophyll in the liquid phase;
e) Contacting the liquid phase of d) with a flocculant and/or an adsorbent, and mixing for a period of time sufficient to flocculate and/or adsorb chlorophyll in the liquid phase to the adsorbent, thereby forming a flocculated mixture;
f) Separating the flocculated mixture of e) into a solid phase and a liquid phase; and
g) Filtering the liquid phase of f) to yield a filtrate containing a purified protein;
   wherein said coagulation in step d) is performed by heating the liquid phase to a first set temperature in no more than about 30 minutes, then cooling it to a second set temperature in no more than about 30 minutes, wherein the cooling is initiated when the liquid phase reaches the first set temperature; and/or by adding a salt to the liquid phase; and/or by using a polymer-based coagulant and/or by electrocoagulation.

As the skilled person will appreciate, said non-denatured RuBisCO may also be obtained by recombinant expression of said protein in a suitable host cell. In such a method, a gene encoding said non-denatured RuBisCO is stably incorporated into the genome of said host cell. Said transformed host cell is cultured in a suitable growth medium to produce said non-denatured RuBisCO, and said non-denatured RuBisCO is isolated from said cell and/or from said culture medium.

In an embodiment, the RuBisCo preparation is obtainable based on a method described in WO2022/226176. The contents of WO2022/226176, in particular the contents regarding the characteristics of the RuBisCo isolate, such as in claims 18-28 thereof are incorporated herein by reference

Preferably said cell is a plant cell or an algae cell, more preferably a cell from duckweed, algae, beetroot, spinach, spinach beet, kale, beet, sugar beet, chard, sea beet, Mangel beet, carrot, sugar beet, corn, pulses, alfalfa, soy or tobacco, more preferably a cell from a leaf thereof.

Alternatively, said cell may be a cell of a micro-organism, such as a yeast, bacterial, fungal or insecticidal cell.

Said RuBisCo protein preparation preferably comprises a protein content of at least 50 wt. %, based on the total dry weight of the protein preparation. More preferably, a protein content of at least 60 wt. %, at least 65 wt. %, at least 70 wt. %, at least 75 wt. %, at least 80 wt. %, at least 85 wt. %, at least 90 wt. %, at least 91 wt. %, at least 92 wt. %, at least 93 wt. %, at least 94 wt. %, at least 95 wt. %, at least 96 wt. %, at least 97 wt. %, at least 98 wt. %, at least 99 wt. %, most preferably 100 wt. %, based on the total dry weight of the protein preparation.

Preferably, a non-denatured RuBisCo preparation comprises between about 50 wt. % and about 100 wt. % of protein, more preferably between about 55 wt. % and about 99 wt. %, between about 60 wt. % and about 98 wt. %, between about 65 wt. % and about 97 wt. %, between about 70 wt. % and about 96 wt. %, between about 75 wt. % and about 95 wt. %, between about 80 and about 90 wt. % of protein, based on the total dry weight of the non-denatured RuBisCo preparation.

In a particularly preferred embodiment, said non-denatured RuBisCo from duckweed has a purity of at least 80 wt. %, based on the total weight of the protein preparation. Such a non-denatured RuBisCo preparation is obtainable using a method as described in any one of Examples 1-4 of WO 2021/00748.

Said non-denatured RuBisCo preparation optionally comprises one or more further plant components, such as cell-wall components, carbohydrates, starch, cellulose, hemicellulose, lignin, pectin, chlorophyll, proteins and lipids, including derivatives thereof.

Said non-denatured RuBisCo preparation further optionally comprises one or more components derived from the process of isolating said non-denatured RuBisCo from a plant or plant part. Examples include, salts, such as salts obtained from buffering an aqueous solution, flocculants, adsorbents, polymers and derivatives thereof.

Examples of salts include salts of sodium, potassium, ammonium, nitrates, acetates, chlorates, perchlorates, sulphates, sulphites, bisulphites, carbonates, phosphates, Tris, (4-(2-hydroxyethyl)-1-piperazineethanesulfonic acid) (HEPES), N-(2-Acetamido)-2-aminoethanesulfonic acid (ACES), 2,2′,2″-Nitrilotriacetic acid (ADA), BES, 2-(N-morpholino) ethanesulfonic acid (MES), (3-(N-morpholino) propanesulfonic acid) (MOPS), 3-morpholino-2-hydroxypropanesulfonic acid (MOPSO), piperazine-N,N′-bis(2-ethanesulfonic acid) (PIPES), 2-{[1,3-Dihydroxy-2-(hydroxymethyl) propan-2-yl]amino}ethane-1-sulfonic acid (TES), and the like.

Preferably, said non-denatured RuBisCo preparation comprises, based on total dry weight of the protein preparation, at most 50 wt. %, at most 40 wt. %, at most 30 wt. %, at most 20 wt. % at most 10 wt. %, more preferably at most 5 wt. %, at most 4 wt. %, at most 3 wt. %, at most 2 wt. % or 1 wt. % of components other than said non-denatured RuBisCo.

Preferably, said RuBisCo preparation comprises between about 50 wt. % and about 100 wt. % of components other than said non-denatured RuBisCo, more preferably between about 55 wt. % and about 99 wt. %, between about 60 wt. % and about 98 wt. %, between about 65 wt. % and about 97 wt. %, between about 70 wt. % and about 96 wt. %, between about 75 wt. % and about 95 wt. %, between about 80 and about 90 wt. % of components other than non-denatured RuBisCo, based on the total dry weight of the non-denatured RuBisCo preparation.

As the skilled person will appreciate, said non-denatured RuBisCo preparation is suitable for use in food products. This means, if further components other than said non-denatured RuBisCo are present, their presence does not contravene legal requirements set by relevant (health) authorities, such as the Food and Drug Administration (FDA) or the European Food Safety Authority (EFSA).

Said batter or dough preferably comprises, based on the total weight of the batter or dough, between about 0.25 wt. % and about 5 wt. % of non-denatured RuBisCo preparation, more preferably between about 0.5 wt. % and about 4 wt. % of non-denatured RuBisCo preparation, more preferably between about 0.7 wt. % and about 3.2 wt. % of non-denatured RuBisCo preparation.

Preferably, said instant food mixture, comprises, based on the total weight of said instant food mixture, between about 0.5 wt. % and about 8 wt. % of non-denatured RuBisCo preparation, more preferably between about 0.7 wt. % and about 6.5 wt. % of non-denatured RuBisCo preparation, more preferably between about 0.9 wt. % and about 4.6 wt. % of non-denatured RuBisCo preparation.

Preferably, said batter, dough or instant food mixture comprises RuBisCo preparation in an amount that is essentially equal to the weight fraction of dried egg white and/or proteinaceous content of liquid egg white present in a comparable reference cake.

### Starch

Said batter, dough or instant food mixture further comprises a starch. Starch advantageously irreversibly gelatinizes in presence of water and at elevated temperature, such as during baking or frying of a batter or dough according to the invention. Upon gelatinization, said starch contributes to providing structure to a food product. Usually, any type of starch is suitable as long as the starch is edible and capable of forming an irreversible gel.

Said starch may be any starch suitable for preparing a batter, dough or instant food mixture according to the invention, preferably a batter, dough or instant food mixture suitable for preparing a sweet bakery product, such as a cake. Preferably said starch comprises wheat starch, corn starch, cassava starch or rice starch, more preferably native wheat starch, native corn starch, native cassava starch, native rice starch or a combination thereof.

Preferably, said batter or dough comprises, based on the total weight of the batter or dough, between about 10 wt. % and about 40 wt. % of starch, preferably wheat starch, more preferably between about 15 wt. % and about 35 wt. % of starch, more preferably between about 20 wt. % and about 30 wt. % of starch.

Preferably, a batter, dough or instant food mixture according to the invention comprises flour as source of starch.

Preferably, said batter, dough or instant food mixture comprises a cereal flour, most preferably said cereal flour comprises wheat flour, spelt flour, corn flour, oat flour, barley flour, rye flour, sorghum flour, buckwheat flour, millet flour, triticale flour, amaranth flour, teff flour, rice flour, quinoa flour and combinations thereof.

Alternatively or additionally, said flour may comprise a non-cereal flour, such as tapioca flour, potato flour, chickpea flour, coconut flour, almond flour, cassava flour, arrowroot flour, or combinations thereof.

Preferably, said batter, dough or instant food mixture comprises wheat flour. Wheat flour typically comprises, based on the total weight of the wheat flour, about 70-75 wt. % of starch, about 14 wt. % of water and about 8-11 wt. % of protein.

As the skilled person will appreciate, any non-denatured RuBisCo that may be present in flour, such as wheat flour, is not calculated in the content of non-denatured RuBisCo preparation, as it is not at least substantially isolated from non-protein components of wheat flour. Any such non-denatured RuBisCo present in flour, are thus added in addition to said non-denatured RuBisCo preparation.

Preferably, said batter or dough according to the invention comprises, based on the total weight of the batter or dough, between about 10 wt. % and about 60 wt. % of flour, preferably wheat flour, more preferably between about 15 wt. % and about 50 wt. % of flour, more preferably between about 20 wt. % and about 45 wt. % of flour.

Preferably, said instant food mixture according to the invention, comprises, based on the total weight of said instant food mixture, between about 20 wt. % and about 75 wt. % of flour, preferably wheat flour, more preferably between about 25 wt. % and about 70 wt. % of flour, more preferably between about 30 wt. % and about 67 wt. % of flour.

Additionally or alternatively, said starch may be provided as food ingredient separate from flour to the batter, dough or instant food mixture according to the invention. This is referred to herein as isolated starch. Isolated starch refers to native starch that has at least been substantially isolated from non-starch components that were present in the natural product it is isolated from.

If starch is added to the batter, dough or instant food mixture in addition to flour, typically said batter, dough or instant food mixture comprises between about 1 wt. % and about 10 wt. % of isolated starch, preferably between about 2 wt. % and about 8 wt. % of isolated starch, more preferably between about 3 and about 7 wt. % of isolated starch.

### Sweetener

Said batter, dough or instant food mixture further comprises a sweetener. Said sweetener may comprise one or more sugars such as glucose, fructose or sucrose, one or more artificial sweeteners, such as aspartame, stevia, maltitol, acesulfame k, sorbitol, one or more oligosaccharides, such as polydextrose, maltodextrin and inulin, or a combination thereof.

Preferably said sweetener comprises a sugar, more preferably sucrose, fructose or glucose, in particular sucrose. Said sugar may be provided in the form of isolated sugar, such as caster sugar or granulated sugar, or may be added in the form of a natural sweetener such as honey, fruit (purée) or the like.

Preferably, said batter or dough according to the invention comprises, based on the total weight of the batter or dough, between about 10 wt. % and about 40 wt. % of sugar, more preferably between about 15 wt. % and about 35 wt. % of sugar, more preferably between about 18 wt. % and about 30 wt. % of sugar.

Preferably, said instant food mixture according to the invention, comprises, based on the total weight of said instant food mixture, between about 30 wt. % and about 55 wt. % of sugar, more preferably between about 35 wt. % and about 50 wt. % of sugar, more preferably between about 40 wt. % and about 45 wt. % of sugar.

### Further Food Ingredients

Said batter, dough or instant food mixture according to the invention further comprises water. Preferably, said batter or dough according to the invention comprises, based on the total weight of said batter or dough, at least 10 wt. % of water, more preferably at least 15 wt. % of water, even more preferably at least 20 wt. % of water, even more preferably at least 25 wt. % of water, most preferably at least 30 wt. % of water.

Preferably, said batter or dough according to the invention further comprises, based on the total weight of said batter or dough, between about 10 wt. % and about 50 wt. % of water, more preferably between about 12 wt. % and about 40 wt. % of water, most preferably between about 15 wt. % and about 36 wt. % of water.

Preferably, said instant food mixture according to the invention comprises, based on the total weight of said instant food mixture, at least 1 wt. % of water, more preferably at least 1.5 wt. % of water, even more preferably at least 2 wt. % of water, even more preferably at least 2.5 wt. % of water, most preferably at least 3 wt. % of water.

Preferably, said instant food mixture according to the invention further comprises, based on the total weight of said food mixture, between about 1 wt. % and about 5 wt. % of water, more preferably between about 1.5 wt. % and about 4 wt. % of water, most preferably between about 3 wt. % and about 3.5 wt. % of water.

Optionally, said batter or dough according to the invention may comprise a lipid. Typically, a batter of dough comprising a lipid is suitable for preparing a fatty food product, preferably a fatty cake, such as a pound cake or a crème cake.

Said lipid may comprise any edible fat or lipid, such as an oil, butter or margarine.

Preferably said lipid comprises a vegetable oil, most preferably rapeseed oil, sunflower oil, walnut oil, olive oil, almond oil, peanut oil, linseed oil, corn oil, safflower oil, soy oil, hazelnut oil, shea oil, palm oil, palm kernel oil, coconut oil, cocoa butter or a fraction thereof. Preferably said oil comprises rapeseed oil or sunflower oil.

Alternatively or additionally, said lipid may comprises butter, lard, shortening (triglycerides, essentially solid at 20° C.) or margarine.

Preferably, said lipid comprises margarine, preferably a vegetable margarine.

Preferably, a batter or dough comprising a lipid according to the invention comprises, based on the total weight of said batter or dough, between about 5 wt. % and about 30 wt. % of a lipid, more preferably between about 10 wt. % and about 30 wt. %, most preferably between about 15 wt. % and about 25 wt. % of a lipid, preferably a vegetable lipid.

Said batter, dough or instant food mixture according to the invention further preferably comprises a leavening salt and/or leavening acid. Leavening salts and leavening acids advantageously produce carbon dioxide, which becomes entrapped into the batter or dough as gas cells, providing a food product comprising a desired volume and texture.

Baking powder, such as bicarbonate salts, preferably sodium bicarbonate and leavening acids such as monocalcium phosphate (MCPO), sodium aluminium phosphate (SALP), sodium aluminium sulphate (SAS), sodium acid pyrophosphate (SAPP) or tartaric acid may be used.

Preferably, said batter, dough or instant food mixture according to the invention comprises at least 1 wt. % of a leavening salt or acid, more preferably at least 1.5 wt. % of a leavening salt or acid, even more preferably at least 2 wt. % of a leavening salt or acid, based on the total weight of batter, dough or instant food mixture.

Preferably, the batter, dough or instant food mixture according to the invention comprises between about 0.5 wt. % and about 4 wt. % of leavening salt or acid, more preferably between about 1 wt. % and about 3.5 wt. % of a leavening salt or acid, even more preferably between about 1.5 wt. % and about 3 wt. % of a leavening salt or acid, based on the total weight of batter, dough or instant food mixture.

Said batter, dough according or instant food mixture to the invention preferably comprises an emulsifier, preferably a lecithin, more preferably a vegetable lecithin. Said lecithin may be present as crude lecithin or as de-oiled lecithin, preferably is present as de-oiled lecithin.

Examples of vegetable lecithin include soy lecithin, sunflower lecithin, rapeseed lecithin, corn lecithin, rice bran lecithin, fava bean lecithin, garbanzo lecithin, canola lecithin, cottonseed lecithin and a combination thereof. Preferably, said batter, dough or instant food mixture according to the invention comprises sunflower lecithin. Preferably said batter, dough or instant food mixture according to the invention is essentially free of egg lecithin.

Without being bound by any theory it is believed that the presence of lecithin further advantageously improves the properties of a batter, dough or instant food mixture according to the invention, and in particular the food product that is prepared from said batter, dough or instant food mixture. In particular, it is believed that the presence of lecithin has a beneficial effect on the volume and texture of said food product and the density of said batter. More in particular, said lecithin advantageously improves the specific cake volume and cake texture of a cake according to the invention compared to a comparable reference cake comprising egg white.

Furthermore, the inventors realized that a batter, dough or instant food mixture comprising lecithin is advantageously suitable to prepare a food product, in particular a cake, with satisfactory properties, in particular good batter density, cake volume, crumb hardness, voidage and mouthfeel, without requiring to use whole egg or egg yolk. This is advantageous, because consumption of egg yolk is typically undesired from health perspective, due to a high content of cholesterol and unsaturated fatty acids.

Preferably, said batter, dough or instant food mixture comprises at least 0.1 wt. % of lecithin, more preferably at least 0.2 wt. % of lecithin, more preferably at least 0.5 wt. % of lecithin, even more preferably at least 0.8 wt. % of lecithin, based on the total weight of batter, dough or instant food mixture.

Preferably, the batter, dough or instant food mixture comprises between about 0.1 wt. % and about 4 wt. % of lecithin, more preferably between about 0.5 wt. % and about 3.5 wt. % of lecithin, even more preferably between about 0.8 wt. % and about 3 wt. % of lecithin, based on the total weight of batter, dough or instant food mixture.

Said batter, dough or instant food mixture according to the invention preferably comprises a RuBisCo preparation and a lecithin, more preferably wherein the weight to weight ratio of RuBisCo preparation content to lecithin content is at least about 1:1, more preferably at least about 3:2, even more preferably at least about 2:1.

In particular, said weight to weight ratio RuBisCo preparation content to lecithin content is in the range of about 1:5 to about 10:1, preferably in the range of about 4:5 to about 5:1, more preferably in the range of about 1:1 to about 5:1, in particular in the range of about 3:2 to about 3:1 or about 3:2 to about 2:1.

Said batter, dough or instant food mixture according to the invention may optionally comprise one or more further emulsifiers, thickener, preservative, salt, a flavouring agent and a colouring agent.

Examples of further emulsifiers include sponge emulsifiers, (distilled) mono- or diglycerides, polyglycerol esters or sodium-stearoyl-lactate (SSL).

Examples of preservatives include sodium and calcium propionate, organic propionates, potassium sorbates and sorbic acid.

Examples of thickeners include gums, such as guar gum and xanthan gum.

Said batter, dough or instant food mixture may further advantageously comprise one or more food ingredients, such as fruits, nuts, sweets, such as pieces of chocolate and syrup and cocoa powder.

In a particularly preferred embodiment, said batter or dough according to the invention, comprises, based on the total weight of the batter or dough, 22-28 wt. % of wheat flour, 24-30 wt. % of sugar, 1.5-4 wt. % of a RuBisCo protein preparation and 30-40 wt. % of water, and optionally 0.5-2 wt. % of de-oiled sunflower lecithin. Such a batter, dough or instant food mixture is particularly suitable for preparing a sponge cake according to the invention.

In another particularly preferred embodiment, said batter or dough according to the invention, comprises, based on the total weight of the batter or dough, 18-24 wt. % of wheat flour, 22-28 wt. % of sugar, 1.2-4 wt. % of a RuBisCo protein preparation and 20-30 wt. % of water, 15-25 wt. % of fat and optionally 0.5-2 wt. % of sunflower lecithin. Such a batter, dough or instant food mixture is particularly suitable for preparing a pound or crème cake according to the invention.

In another particularly preferred embodiment, said batter or dough according to the invention, comprises, based on the total weight of the batter or dough, 35-45 wt. % of wheat flour, 17-21 wt. % of sugar, 0.5-2 wt. % of a RuBisCo protein preparation and 25-35 wt. % of water, 5-10 wt. % of fat and optionally 0.5-2 wt. % of sunflower lecithin. Such a batter, dough or instant food mixture is particularly suitable for preparing a cake doughnut according to the invention.

A batter, dough or instant food mixture that is essentially free of eggs can be used to prepare a food product that is suitable for consumers that have an allergy against eggs.

Accordingly, said batter, dough or instant food mixture preferably comprises, based on the total weight of batter, at most 30 wt. % of whole liquid egg, more preferably at most 20 wt. % of whole liquid egg, at most 10 wt. % of whole liquid egg, at most 8 wt. % of whole liquid egg, at most 5 wt. % of whole liquid egg, at most 3 wt. % of whole liquid egg at most 2 wt. % of whole liquid egg, at most 1 wt. % of whole liquid egg, most preferably is essentially free of whole liquid egg.

As the skilled person will appreciate said batter, dough or instant food mixture according to the invention can further be used to prepare a food product that is essentially free of animal derived products.

Accordingly, the invention preferably relates to a batter, dough or instant food mixture that is vegan.

### Heated Food Product

The invention further relates to a heated food product, comprising a heated dough or batter according to the invention.

Without wishing to be bound by any theory, it is believed that during a baking, frying, cooking or steaming process, the non-denatured RuBisCo present in the batter or dough according to the invention, becomes denatured, thereby forming an aggregate that functions as a binder of other food ingredients, in particular sweetener and flour. Typically, said denatured RuBisCo forms an at least substantially continuous phase, having further food ingredients dispersed therein.

It has been advantageously found that said denatured RuBisCo provides sufficient strength to the food product, such that the presence of egg—in particular the proteinaceous matter present in egg white—is no longer required.

Accordingly, the invention further relates to a heated food product, comprising a denatured RuBisCo preparation, a starch, a sweetener and water, and optionally a lipid. Said sweetener, starch, water and lipid are as described herein above. Said denatured RuBisCo preparation is obtained by heating a batter or dough comprising a non-denatured RuBisCo as defined herein above, above the denaturing temperature of said non-denatured RuBisCo preparation.

Preferably, said heated food product according to the invention comprises, based on the total weight of the food product, between about 25-40 wt. % of wheat flour, about 20-35 wt. % of sugar, about 1.5-5 wt. % of a RuBisCo protein preparation, about 15-25 wt. % of water, and optionally about 0.5-2 wt. % of lecithin, preferably (de-oiled) sunflower lecithin.

In another particularly preferred embodiment, said heated food product according to the invention, comprises, based on the total weight of the food product, about 20-45 wt. % of wheat flour, about 15-25 wt. % of sugar, about 0.7-4 wt. % of a RuBisCo protein preparation, about 5-14 wt. % of water, about 5-20 wt. % of a lipid and optionally about 0.5-2 wt. % of lecithin, preferably (de-oiled) sunflower lecithin.

Said heated food product further preferably comprises a RuBisCo protein preparation and a lecithin, preferably wherein the lecithin comprises or essentially consists of one or more vegetable lecithin. More preferably wherein the weight to weight ratio of the RuBisCo preparation content to lecithin content is at least about 1:1, more preferably at least about 3:2, even more preferably at least about 2:1. In particular wherein the weight to weight ratio of the RuBisCo preparation content to lecithin content is in the range of about 1:5 to about 10:1, preferably in the range of about 4:5 to about 5:1, more preferably in the range of about 1:1 to about 5:1, in particular in the range of about 3:2 to about 3:1 or about 3:2 to about 2:1.

Preferably, said heated food product according to the invention comprises a RuBisCo preparation content between about 0.5 wt. % and about 8 wt. %, based on total ingredients other than water, and a lecithin content, which lecithin content preferably comprises or essentially consists of vegetable lecithin, between about 0.1 and about 4 wt. %, based on total ingredients other than water; in particular comprises a RuBisCo preparation content between about 0.7 wt. % and about 6.5 wt. %, based on total ingredients other than water, and a lecithin content, which lecithin content preferably comprises or essentially consists of vegetable lecithin, between about 0.5 and about 3.5 wt. %, based on total ingredients other than water; more in particular comprises a RuBisCo preparation content between about 0.9 wt. % and about 4.6 wt. %, based on total ingredients other than water, and a lecithin content, which lecithin content preferably comprises or essentially consists of vegetable lecithin, between about 0.8 and about 3 wt. %, based on total ingredients other than water.

Said heated food product may in principle be any food product that may be obtained by baking, frying, cooking or steaming a batter or dough according to the invention, which is classically prepared with egg or a fraction thereof, but is preferably a food product having a specific void volume in excess of 50 vol. %. Preferably, said heated food product is a baked or fried food product. A baked food product is typically prepared by dry heat, e.g. using a convection oven. A fried food product is typically prepared by using a liquid oil, e.g. by contacting (e.g. pan frying) or immersing (e.g. deep frying) said food product in a hot liquid oil, typically at a temperature of at least 150° C.

Preferably, said heated food product has a void volume of at least 50 vol. %, more preferably at least 60 vol. %, at least 70 vol. %, more preferably at least 75 vol. %, at least 80 vol. %, at least 85 vol. % at least 90 vol. % as determinable with a X-ray microCT (Bruker 1172 microCT instrument) using the method as described in Example 1.

Preferably, said heated food product has a void volume of between about 50 vol. % and about 90 vol. %, more preferably between 55 vol. % and 70 vol. %.

Said heated food product according to the invention is preferably a cake. Examples of cakes with a void volume of at least 50 vol. % include sponge cakes, pound cakes, cake muffins, cupcakes, crème cakes, genoese cakes, chiffon cakes, angel cakes and cake doughnuts. Preferably said cake is a sponge cake, a pound cake, a cupcake, a cake muffin or a cake doughnut.

A heated food product according to the invention that is essentially free of food ingredients of animal origin is suitable for consumers that follow a vegan diet, and for consumers that have an allergy against one or more food ingredients of animal origin, such as consumers with an allergy against egg.

Accordingly, said heated food product according to the invention preferably comprises, based on the total weight of proteinaceous matter, at most 3 wt. % of egg white protein, more preferably at most 2 wt. % of egg white, at most 1 wt. % of egg white, most preferably is essentially free of egg white.

Accordingly, the invention preferably relates to a heated food product that is vegan.

Said heated food product, preferably said cake may comprise one or more further food ingredient, for instance incorporated in the heated batter or dough, such as fruit, including dried fruit, nuts, sweets, custard, and chocolate or present as a topping or icing, such as cream cheese, glaze, syrup, jam, cream and the like.

The invention further preferably relates to a sponge cake having a voidage of between about 67 vol. % and about 90 vol. % as determinable with a X-ray microCT (Bruker 1172 microCT instrument) using the method as described in Example 1 and/or a specific volume of at least about 3.5 g/ml more preferably at least 4 g/ml as determinable with a “Volscan” from Stable Micro Systems Ltd using a method as described in Example 1 and/or a crumb hardness of at least about 310 g, more preferably at least 400 g as determinable with a texture analyser (TA.XTplusC) from Stable Micro Systems Ltd using the method as described in Example 1.

The invention further preferably relates to a pound cake having a voidage of between about 60 vol. % and about 90 vol. % and/or a specific volume of at least about 1.6 g/ml more preferably at least 2 g/ml and/or a crumb hardness of at least about 500 g, more preferably at least 1000 g.

The invention further preferably relates to a cake doughnut having a voidage of between about 50 vol. % and about 90 vol. % and/or a specific volume of at least about 2.0 g/ml and/or a crumb hardness of at least about 8000 g.

Preferably, the invention relates to a heated food product that has similar cake properties, in particular a similar crumb hardness compared to a comparable reference cake comprising egg. Preferably, the crumb hardness is at most 20% higher or lower, more preferably at most 15%, even more preferably at most 10% higher or lower than the crumb hardness of said reference cake.

The skilled person is able to select a suitable comparable reference cake. Preferably, a reference cake comprising egg white instead of the RuBisCo preparation, but of which the composition is otherwise the same. More preferably, the weight amount of dried egg white and/or proteinaceous content of liquid egg white in the reference cake is substantially the same as the weight amount of RuBisCo in the cake according to the invention.

The invention further preferably relates to a heated food product, preferably a baked or fried food product that is obtainable by a method according to the invention.

### Method/Use

The invention further relates to a method for preparing an instant food mixture according to the invention, comprising mixing a non-denatured RuBisCo, a sweetener and a starch.

The invention further relates to a method for preparing a batter or a dough according to the invention, comprising mixing a non-denatured RuBisCo, a sweetener, a starch and water, or mixing an instant food mixture with water, to obtain a batter or a dough according to the invention.

Said non-denatured RuBisCo, said sweetener, said starch and said water are as described herein above.

Preferably, said method for preparing a batter or a dough further comprises mixing one or more of a lipid, a leavening salt or leavening acid, a lecithin, a further emulsifier, a preservative, a flavouring agent, or a colouring agent.

Said mixing may be achieved using any suitable method known in the art, such as beating, whisking or mechanical mixing.

The invention further preferably relates to a method for preparing a heated food product, preferably a cake, baking, frying, steaming or cooking said batter or dough according to the invention at a temperature above the denaturing temperature of said non-denatured RuBisCo to obtain a heated food product according to the invention.

Typically, said batter or dough according to the invention is heated for a time sufficient to denature said non-denatured RuBisCo, to gelatinize the starch and to pasteurize the batter or dough throughout-such that the inner-part reaches a temperature typically of 90° C. or more. As the skilled person will understand, the term “inner part” refers to a location in the (heated) batter or dough that is at least approximately 10% removed from the outer of the total dimension of the shaped batter or dough, in particular the core of the batter of dough. For example, if the baking tin holding the batter has a length of 15 cm, width of 7 cm and a height of 7 cm, than an inner part is at least approximately 1.5 cm removed of outer surface when measured over the length, approximately 0.7 cm removed from the outer surface when measured of the width and height.

The time-temperature function is dependent on the mass of the cake and heat transfer, as understood by a person skilled in the art of baking.

For a batter or dough according to the invention of approximately 1 kg and preferably in dimensions as indicated in Examples 1 and 2, an outer surface of said batter or dough is typically heated to a temperature above 50° C., more preferably at a temperature above 80° C., a temperature above 100° C., a temperature above 120° C., a temperature above 140° C., a temperature above 160° C., most preferably a temperature above 180° C.

Preferably, said outer surface of said batter or dough according to the invention is heated to a temperature of between about 50° C. and about 250° C., more preferably between about 80° C. and about 220° C., even more preferably between about 100° C. and about 200° C., most preferably between about 150° C. and about 190° C.

Said batter or dough is heated using any suitable means known in the art, such as by baking, frying, cooking or steaming.

Preferably, said batter or dough is heated by baking or frying. Baking or frying typically refers to heating in absence of substantial amounts of water, such as by using dry heat (e.g. in the oven) or by using oil (e.g. in a fryer).

In principle, said batter or dough may be baked or fried using any suitable means known in the art, but is preferably baked in an oven.

Alternatively, said batter or dough may be cooking or steaming, i.e. in presence of water. Cooking or steaming may be achieved using any suitable means known in the art, such as in a cooking pan or in a steaming oven.

The invention further relates to a use of non-denatured RuBisCo preparation as an egg substitute or egg white substitute in a food product, preferably a heated food product, more preferably a cake, a dough or a batter.

The invention further relates to a use of non-denatured RuBisCo preparation as a binder in a food product, preferably a heated food product, more preferably a cake, a dough or a batter.

For the purpose of clarity and a concise description, features are described herein as part of the same or separate embodiments, however, it will be appreciated that the scope of the invention may include embodiments having combinations of all or some of the features described.

The invention is demonstrated by the following examples.

### EXAMPLES

### Example 1: Method for Making Sponge Cake

A recipe for a reference fat-free sponge cake is shown in Table 1. It can be useful to separate the egg into yolk and albumen, including dry egg white, and the recipe of Table 1 would then be reformulated to that of Table 2. An advantage of using dry egg white in a reference formulation is that direct comparison can then be made to non-egg proteins. Recipes for sponge cakes according to the invention are shown in Table 3.

TABLE 1
reference sponge cake recipe - using whole egg

| Ingredient | supply examples | g | wt. % |
|---|---|---|---|
| Cake wheat flour | Flamingo from Meneba | 250 | 26.0 |
| Sugar (granulated) | | 265 | 27.6 |
| Wheat starch | Native starch from Roquette | 15 | 1.6 |
| Sponge emulsifier | Emulpals 110 from Palsgaard | 25 | 2.6 |
| SAPP 15 | Levall AR15 from Budenheim | 10 | 1.04 |
| Sodium | | 7.3 | 0.76 |
| bicarbonate | | | |
| Salt | | 1.0 | 0.10 |
| Potassium sorbate | | 2.0 | 0.21 |
| Whole liquid egg | Pasteurised | 385 | 40.0 |

| | | |
|---|---|---|
| Total | 960.3 | 100 |

TABLE 2
reference sponge cake recipe - using liquid
egg yolk and dried egg white powder

| Ingredient | supply examples | g | wt. % |
|---|---|---|---|
| Cake wheat flour | Flamingo from Meneba | 250 | 26.0 |
| Sugar (granulated) | | 265 | 27.6 |
| Wheat starch | Native starch from Roquette | 15 | 1.6 |
| Sponge emulsifier | Emulpals 110 from Palsgaard | 25 | 2.6 |
| SAPP 15 | Levall AR15 from Budenheim | 10 | 1.04 |
| Sodium | | 7.3 | 0.76 |
| bicarbonate | | | |
| Salt | | 1.0 | 0.10 |
| Potassium sorbate | | 2.0 | 0.21 |
| Dried egg white | Powder from Cocovite | 20 | 2.1 |
| Liquid egg yolk | Pasteurised | 85 | 8.9 |
| Water | | 280 | 29.1 |

| | | |
|---|---|---|
| Total | 960.3 | 100 |

Dry ingredients were preblended and placed in a Hobart mixing bowl (5 L). Water and any liquid egg inclusion were added to the dry ingredients. Using a whisking attachment, batter was mixed slowly, speed 1, for 1 minute, before increasing to speed 3 for 3 minutes. The bowl was scraped down, then, mixing continued for a further 3 minutes at speed 3. The batter was mixed for 1 further minute at speed 1, before 300 g portions were transferred to round cake tins (22 cm top diameter, 19 cm bottom diameter 4.5 cm deep, coated with release agent). The batter was smoothed with a suitable spatula and then baked in a deck oven for 19 minutes at 185° C. After baking, the cake was removed from the tin and allowed to cool down to room temperature.

The cakes were evaluated in terms of batter density, cake specific volume, crumb hardness, cake voidage and mouthfeel.

The density of batter was calculated from the weight of batter levelled in a bowl of 100 ml.

A “Volscan” from Stable Micro Systems Ltd was used for measurements of specific volume—this equipment being commonly used in test bakeries. The Volscan measures volume through laser scanning—in this instance we used settings for the incremental at 3 mm and a rotation speed of 1 revolution per second.

To measure crumb hardness, a texture analyser (TA.XTplusC) from Stable Micro Systems Ltd was used using sponge cakes one day after baking. Products were stored in impermeable bags until they were measured to prevent them from drying. Crumb hardness was the maximum force recorded from a 30 kg load cell, using 100 mm probe depressed at rate of 1 mm/s to depth to a depth 10 mm into a cake slice. The cake slices were 41 mm deep and 49 mm×49 mm w/l—with measurements being averaged from four slices and two cakes.

Measurements of voidage (or void volume) have been made for some of the cakes using X-ray microCT. This is a non-destructive method for obtaining 3D images of an internal structure. Prepared samples were mounted inside a Bruker 1172 microCT instrument. Transmission images were acquired with a 1 mm Al filter. The source power was 50 kV and 100 μA. In order to minimise noise, 4×4 pixel binning was used. Images were subsequently reconstructed and analysed using NRecon and CT Analyser software respectively (Bruker). The resolution of the reconstructed images was 34.64 μm (pixel size). A sample of the sponge was cut from an off-centre position-without external crust- and the cut surfaces were digitally removed (ca 1 mm) to give a measurement judged to be representative of the internal structure.

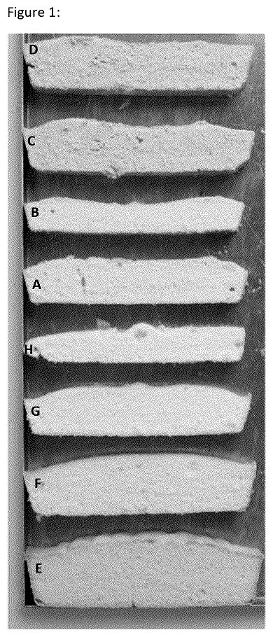

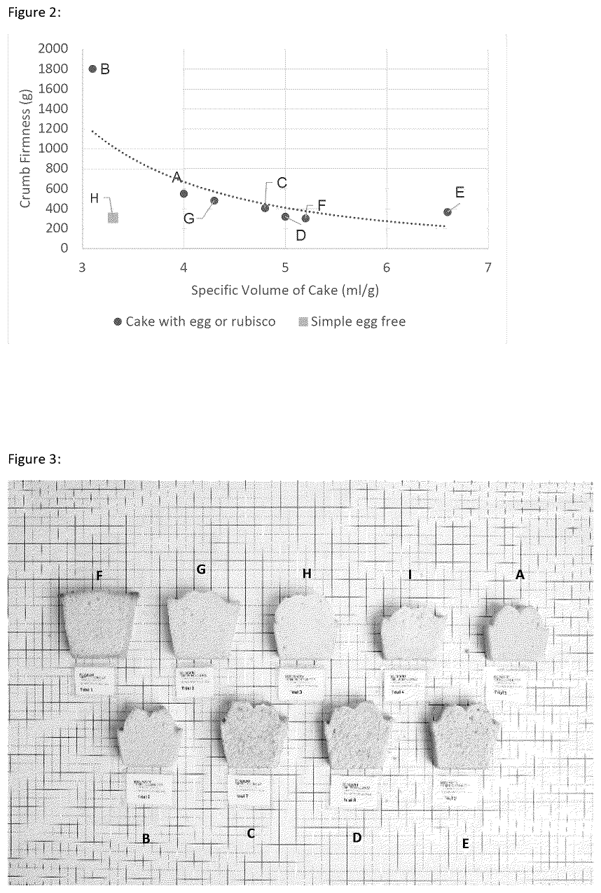

The results are shown in Table 3 and FIGS. 1 and 2.

TABLE 3
Examples of sponge cake recipes according to the invention against references;

| | | Cake F | | | | | | Cake D |
|---|---|---|---|---|---|---|---|---|
| | | Ref. | | | | | | Egg |
| | Cake E | Liquid | Cake G | | | | Cake C | Free + |
| | Ref. | yolk | Ref. | | Cake A | Cake B | Egg | RP#1 + |
| | Whole | and | Dried | Cake H | Egg | Egg | Free + | Lecithin + |
| | liquid | dried | white | Ref. Egg | Free + | Free + | RP#1 + | Adjusted |
| Ingredient | egg | white | only | Free | RP#1 | RP#1.5 | Lecithin | Leavening |
| Cake wheat flour | 26.0 | 26.0 | 26.0 | 26.0 | 26.0 | 26.0 | 26.0 | 26.0 |
| Sugar (granulated) | 27.7 | 27.7 | 27.7 | 27.7 | 27.7 | 27.7 | 27.7 | 27.7 |
| Wheat starch | 1.6 | 1.6 | 6.2 | 8.3 | 6.2 | 5.2 | 5.1 | 4.5 |
| Sponge emulsifier | 2.6 | 2.6 | 2.6 | 2.6 | 2.6 | 2.6 | 2.6 | 2.6 |
| SAPP 15 | 1.04 | 1.04 | 1.04 | 1.04 | 1.04 | 1.04 | 1.04 | 1.40 |
| Sodium bicarbonate | 0.76 | 0.76 | 0.76 | 0.76 | 0.76 | 0.76 | 0.76 | 1.0 |
| Salt | 0.10 | 0.10 | 0.10 | 0.10 | 0.10 | 0.10 | 0.10 | 0.10 |
| Potassium sorbate | 0.21 | 0.21 | 0.21 | 0.21 | 0.21 | 0.21 | 0.21 | 0.21 |
| Dried egg white | | 2.1 | 2.1 | | | | | |
| Rubisco Protein | | | | | 2.1 | 3.2 | 2.1 | 2.1 |
| Whole liquid egg | 40.0 | | | | | | | |
| Liquid egg yolk | | 8.9 | | | | | | |
| Water | | 29.0 | 33.3 | 33.2 | 33.4 | 33.4 | 33.4 | 33.4 |
| Sunflower Lecithin | | | | | | | 1.0 | 1.0 |
| Total | 100 | 100 | 100 | 100 | 100 | 100 | 100 | 100 |
| Batter density (g/ml) | 0.51 | 0.41 | 0.46 | 0.45 | 0.41 | 0.44 | 0.54 | 0.52 |
| Cake specific vol. (ml/g) | 6.6 | 5.2 | 4.3 | 3.3 | 4.0 | 3.1 | 4.8 | 5.0 |
| Crumb hardness (g) | 363 | 301 | 479 | 308 | 547 | 1799 | 403 | 318 |
| Cake voidage (%) | N/T | 78.6 | 70.5 | 66.6 | 68.5 | N/T | 72.7 | N/T |
| Mouthfeel | Firm and | Firm and | Firm and | Soft and | Firm and | Very firm | Firm | Firm |
| | cohesive | cohesive | cohesive | fragile | cohesive | and | and | and |
| | | | | (unacceptable) | | cohesive | cohesive | cohesive |

# RuBisCo protein was supplied from Plantible Foods, Inc. of protein having a purity of at least 80 wt. %, based on the total weight of the protein preparation;
* Sunflower Lecithin was deoiled lecithin from Cargill Foods; N/T measurement not made; RP = RuBisCo Protein.

It will be seen that Reference cakes E, F and G had specific cake volume in the range of 4.3-6.6 ml/g, or voidages in excess of 70.5%. These sponge cakes were regarded as being firm and cohesive—with a crumb hardness in excess of 301 g (by texture analysis). When all egg components were removed (Reference cake H) the specific cake volume was lower (3.3 ml/g) and, in spite of this loss of volume, crumb hardness (308 g) was not substantially greater than those with egg. The mouthfeel of this sponge cake was regarded as being soft and fragile.

When RuBisCo protein was added to the egg free recipe, a lower addition level (RP #1) gave increased volume and crumb hardness; a higher addition level (RP #1.5) gave much increased crumb hardness (cakes A and B respectively).

Use of lecithin and adjusted leavening allowed a more complete replacement of egg, giving a greater specific volume while maintaining a satisfactory crumb firmness (cakes C and D). By mouthfeel, the crumb of all cakes with RuBisCo were regarded as firm and cohesive- and of a similar textural quality to sponges containing egg.

The functional qualities of RuBisCo against egg references in sponge cake are highlighted with FIG. 2. As cake volume increases, crumb hardness typically would be expected to fall-simply because there is a more porous structure. We see a curve that broadly follows this principal for sponge variants made containing egg or RuBisCo. There is but one outlier: the egg-free sponge, free also of RuBisCo (Ref D). Here, both volume and crumb hardness are decreased.

It is concluded that RuBisCo can serve as an egg replacer in sponge cake.

### Example 2: Method for Making Pound and Creme Cake

Recipe for a reference pound and creme cake is shown in Table 4. It can be useful to separate the egg into yolk and albumen, including dry egg white, and the recipe of Table 4 would then be reformulated to that of Table 5. An advantage of using dry egg white in the reference formulation is that direct comparison can then be made to non-egg proteins.

TABLE 4
reference pound and creme cake recipe - using whole egg

| Ingredient | supply examples | g | wt. % |
|---|---|---|---|
| Cake wheat flour | Albatros from Meneba | 250 | 25.6 |
| Sugar (caster) | | 250 | 25.6 |
| Distilled monoglyceride | Dimodan from Danisco | 3 | 0.31 |
| SAPP 28 | | 12.8 | 1.31 |
| Sodium bicarbonate | | 8 | 0.82 |
| Salt | | 2.5 | 0.26 |
| Rapeseed oil or Margarine | | 200 | 20.5 |
| Whole liquid egg | Pasteurized | 250 | 25.6 |

| | | |
|---|---|---|
| Total | 976.3 | 100 |

TABLE 5
reference pound and creme cake recipe - using
liquid egg yolk and dried egg white powder

| Ingredient | supply examples | g | wt. % |
|---|---|---|---|
| Cake wheat flour | Albatros from Meneba | 250 | 25.6 |
| Sugar (caster) | | 250 | 25.6 |
| Distilled monoglyceride | Dimodan from Danisco | 3 | 0.31 |
| SAPP 28 | | 12.8 | 1.31 |
| Sodium bicarbonate | | 8 | 0.82 |
| Salt | | 2.5 | 0.26 |
| Dried egg white | | 15 | 1.54 |
| Rapeseed oil or Margarine | | 200 | 20.5 |
| Liquid egg yolk | Pasteurized | 85 | 8.71 |
| Water | | 150 | 15.4 |

| | | |
|---|---|---|
| Total | 976.3 | 100 |

Dry ingredients were preblended and placed in a Hobart mixing bowl (5 L) together with oil/margarine. Water and any liquid egg inclusion were added to the dry ingredients. Using a beater attachment, batter was mixed slowly, speed 1, for 1 minute, before increasing to speed 2 for 3 minutes. The bowl was scraped down, then, mixing continued for a further 1 minute at speed 1. Portions of 300 g were transferred to loaf tins (17 cm long×9 cm wide at the top, 15 cm long and 6 cm wide at the bottom, 7 cm deep, coated with release agent). The batter was smoothed with a suitable spatula and then baked in a deck oven for 35 minutes at 185° C. After baking, the cake was removed from the tin and allowed to cool down.

The cakes were evaluated in terms of batter density, specific cake volume crumb hardness, voidage and mouthfeel as described in Example 1.

For measurements of voidage, a sample of the cake was cut from a central position-without external crust- and the cut surfaces were digitally removed (ca 1 mm) to give a measurement judged to be representative of the internal structure.

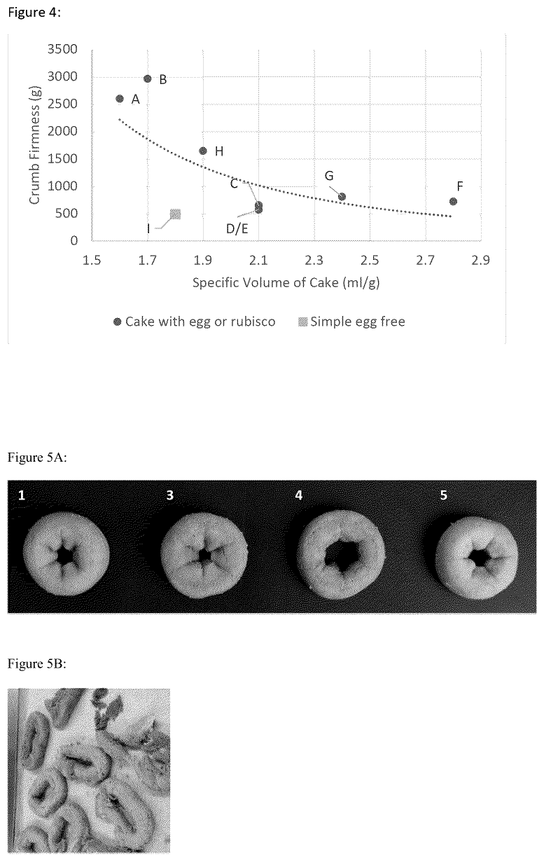

The results are shown in Table 6 and FIGS. 3 and 4.

TABLE 6
pound and crème cakes according to the invention against references;

| | | Cake G | | | | | | | Cake E |
|---|---|---|---|---|---|---|---|---|---|
| | | Ref. | | | | | | Cake D | Egg Free |
| | Cake F | Liquid | Cake H | | | | Cake C | Egg Free | RP#1 + |
| | Ref. | yolk | Ref. | Cake I | Cake A | Cake B | Egg | RP#1 + | Lecithin + |
| | Whole | and | Dried | Ref. | Egg | Egg | Free | Lecithin + | Adjusted |
| | liquid | dried | white | Egg | Free | Free | RP#1 + | Adjusted | Leavening |
| Ingredient | egg | white | only | Free | RP#1 | RP#1.5 | Lecithin | Leavening | No starch |
| Cake wheat flour | 24.4 | 24.4 | 24.4 | 24.4 | 24.4 | 24.4 | 24.4 | 24.4 | 28.2 |
| Sugar (caster) | 24.4 | 24.4 | 24.4 | 24.4 | 24.4 | 24.4 | 24.4 | 24.4 | 24.4 |
| Wheat starch | | | 4.4 | 5.9 | 4.4 | 3.7 | 4.4 | 3.8 | |
| Distilled monoglyceride | 0.29 | 0.29 | 0.29 | 0.29 | 0.29 | 0.29 | 0.29 | 0.29 | 0.29 |
| SAPP 28 | 0.98 | 0.98 | 0.98 | 0.98 | 0.98 | 0.98 | 1.34 | 1.34 | 0.98 |
| Sodium bicarbonate | 0.68 | 0.68 | 0.68 | 0.68 | 0.68 | 0.68 | 0.94 | 0.94 | 0.68 |
| Salt | 0.24 | 0.24 | 0.24 | 0.24 | 0.24 | 0.24 | 0.24 | 0.24 | 0.24 |
| Dried egg white | | 1.47 | 1.47 | | | | | | |
| RuBisCo Protein | | | | | 1.47 | 2.20 | 1.47 | 1.47 | 1.47 |
| Rapeseed Oil | 19.6 | 19.6 | 19.6 | 19.6 | 19.6 | 19.6 | 18.6 | 18.6 | 18.6 |
| Sunflower Lecithin | | | | | | | 1.0 | 1.0 | 1.0 |
| Whole liquid egg | 24.4 | | | | | | | | |
| Liquid egg yolk | | 8.3 | | | | | | | |
| Water | 4.9 | 19.6 | 23.5 | 23.5 | 23.5 | 23.5 | 23.5 | 23.5 | 23.5 |
| Total | 100 | 100 | 100 | 100 | 100 | 100 | 100 | 100 | 100 |
| Batter density (g/ml) | 1.03 | 1.08 | 1.12 | 1.16 | 1.12 | 1.12 | 1.09 | 1.09 | 1.09 |
| Cake specific vol. (ml/g) | 2.8 | 2.4 | 1.9 | 1.8 | 1.6 | 1.7 | 2.1 | 2.1 | 2.1 |
| Crumb hardness (g) | 719 | 806 | 1644 | 490 | 2599 | 2962 | 639 | 569 | 650 |
| Voidage (%) | N/T | 62.6 | 57.3 | 53.8 | 57.3 | N/T | 61.5 | N/T | N/T |
| Mouthfeel | Firm & | Firm & | Firm & | Fragile | Very | Very | Firm & | Firm & | Firm & |
| | cohesive | cohesive | cohesive | & Soft | firm & | firm & | cohesive | cohesive | cohesive |
| | | | | (unaccept.) | cohesive | cohesive | | | |

# RuBisCo protein preparation was supplied from Plantible Foods, Inc., of protein having a purity of at least of 80 wt. %, based on total weight of the protein preparation;
* Sunflower Lecithin was deoiled lecithin from Cargill Foods; N/T measurement not taken; RP = RuBisCo Protein.

It will be seen that Reference cakes F, G and H had specific cake volume in the range of

1.9-2.8 ml/g, or voidages in excess of 57.3%. These crème cakes had a crumb firmness in the range 719-1644 g by texture analysis and were regarded as being firm and cohesive when eaten. When all egg components were removed, Reference cake I, the specific cake volume was lower (1.8 ml/g) and, in spite of the loss of volume, the crumb firmness was only 490 g. The mouthfeel of this crème cake was soft and fragile.

When RuBisCo protein was added to the egg free recipe (cakes A and B), specific volume appeared not to have been improved but, because void volume was increased, we can presume differences in shape have compromised the reliability of specific volume; certainly crumb firmness was markedly increased with the use of RuBisCo relative to Reference I.

Use of lecithin and adjusted leavening allowed an even more complete replacement of egg, giving specific volume and crumb firmness similar to cakes containing egg components (cakes C and D). Starch was used as a filler with most of the reformulations, but in one instance flour was used to maintain solids (Cake E). This cake gave very similar results to its counterpart with starch (Cake D).

By mouthfeel, the crumb of all cakes with RuBisCo were regarded as firm and cohesive- and of a similar textural quality to sponges containing egg.

The functional qualities of RuBisCo against egg references in sponge cake are highlighted with FIG. 4. As cake volume increases, crumb hardness typically would be expected to fall-simply because there is a more porous structure. We see a curve that broadly follows this principal for sponge variants made containing egg or rubisco. There is but one outlier: the egg-free sponge, free also of RuBisCo. Here, both volume and crumb hardness are decreased.

It is concluded that RuBisCo can serve as an egg replacer in pound or creme cake.

### Example 3: Method for Making Cake Doughnut

A recipe for a cake doughnut is shown in Table 7 which makes use of whole dried egg. It can be useful to evaluate dried egg white separately, and the recipe of Table 7 would then be reformulated to that of Table 8. An advantage of using dried egg white in a reference formulation is that comparison can then be made to dry non-egg proteins.

TABLE 7
Basic cake doughnut recipe - using dried whole egg

| Ingredient | Supply examples | g | % |
|---|---|---|---|
| Bread wheat flour | Kolibri from Meneba | 660 | 43.4 |
| Wheat Starch | Native starch from | 15 | 1.0 |
| | Roquette | | |
| Dextrose Monohydrate | General commodity | 15 | 1.0 |
| Sugar (granulated) | General commodity | 290 | 19.1 |
| Sodium bicarbonate | Budenheim | 11 | 0.72 |
| SAPP 28 | Budenheim | 13.75 | 0.90 |
| Lecithin | Cargill, De-oiled Sunflower | 6 | 0.39 |
| Whole dried egg powder | Igreca | 37.5 | 2.5 |
| Salt | General commodity | 3.5 | 0.23 |
| CMC Gum | IFF-Dow | 2 | 0.13 |
| Potassium Sorbate | General commodity | 1.5 | 0.10 |
| Margarine | General commodity | 85 | 5.6 |
| Water | | 380 | 25 |

| | | |
|---|---|---|
| Total | 1520.3 | 100 |

TABLE 8
Basic cake doughnut recipe - using dried egg white

| Ingredient | Supply examples | g | % |
|---|---|---|---|
| Bread wheat flour | Kolibri from Meneba | 660 | 43.4 |
| Wheat Starch | Native starch from | 25.5 | 1.7 |
| | Roquette | | |
| Dextrose Monohydrate | General commodity | 15 | 1.0 |
| Sugar (granulated) | General commodity | 290 | 19.1 |
| Sodium bicarbonate | Budenheim | 11 | 0.72 |
| SAPP 28 | Budenheim | 13.75 | 0.90 |
| Lecithin | Cargill, De-oiled Sunflower | 12.0 | 0.79 |
| Dried egg white | From Bakeplus | 11.0 | 0.72 |
| Salt | General commodity | 3.5 | 0.23 |
| CMC Gum | IFF-Dow | 2 | 0.13 |
| Potassium Sorbate | General commodity | 1.5 | 0.10 |
| Margarine | General commodity | 100 | 6.6 |
| Water | | 375 | 24.7 |

| | | |
|---|---|---|
| Total | 1520.3 | 100 |

Dry ingredients and the margarine were blended together in a Hobart mixing bowl (5 L) for 1 minute at speed 1-using a whisking attachment. Water was added and mixing continued for 1 minute at speed 1 and then, after scraping the bowl down, the batter was mixed for a further 2.5 minutes at speed 2. The batter was allowed to stand for 10 min. Ring doughnuts were formed from approximately 45 g batter using a depositor, frying these in oil at 190 C for 2 min 15 s, flipping these over halfway through the frying.

The cakes were evaluated in terms of batter density, specific cake volume crumb hardness, voidage and mouthfeel as described in Example 1.

When measuring crumb hardness, doughnuts were used three days from being fried. Doughnuts were halved through the ring of the doughnut and the probe pressed onto the flat cut surface.

For measurements of voidage, a sample of the doughnut approximately 60 mm wide was cut from each doughnut and the whole sample was analysed.

The results are shown in Table 9 and FIG. 5.

TABLE 9
cake doughnuts according to the invention against references; # RuBisCo
protein preparation was supplied from Plantible Foods, Inc., of protein having
a purity of at least of 80 wt. %, based on total weight of the protein preparation;

| | | | Trial 3 | | |
|---|---|---|---|---|---|
| | Trial 1 (Ref.) | Trial 2 | (Ref.) | Trial 4 | Trial 5 (Ref.) |
| | Whole dried | (Ref.) | Dried Egg | Egg Free + | Whole dried |
| Ingredient | egg (no SMP) | Egg Free | white | RP#1 | egg with SMP |
| Bread flour | 43.4 | 43.4 | 43.4 | 43.4 | 43.4 |
| Wheat starch | 1.0 | 2.4 | 1.7 | 1.7 | |
| Dextrose | 1.0 | 1.0 | 1.0 | 1.0 | |
| monohydrate | | | | | |
| Sugar | 19.1 | 19.1 | 19.1 | 19.1 | 19.1 |
| (granulated) | | | | | |
| Sodium | 0.72 | 0.72 | 0.72 | 0.72 | 0.72 |
| bicarbonate | | | | | |
| SAPP 28 | 0.90 | 0.90 | 0.90 | 0.90 | 0.90 |
| Dried egg white | | | 0.72 | | |
| RuBisCo | | | | 0.72 | |
| Sunflower | 0.39 | 0.79 | 0.79 | 0.79 | 0.39 |
| Lecithin | | | | | |
| Whole dried | 2.47 | | | | 2.47 |
| egg | | | | | |
| Salt | 0.23 | 0.23 | 0.23 | 0.23 | 0.23 |
| Skimmed milk | | | | | 2.0 |
| powder (SMP) | | | | | |
| CMC Gum | 0.13 | 0.13 | 0.13 | 0.13 | 0.13 |
| Potassium | 0.10 | 0.10 | 0.10 | 0.10 | 0.10 |
| sorbate | | | | | |
| Water | 25.0 | 24.7 | 24.7 | 24.7 | 25.0 |
| Margarine | 5.6 | 6.6 | 6.6 | 6.6 | 5.6 |
| Total | 100 | 100 | 100 | 100 | 100 |
| Cake volume | 2.3 | N/A | 2.0 | 2.0 | 2.3 |
| (ml/g) | | | | | |
| Crumb | 8023 | N/A | 7400 | 11339 | 8739 |
| hardness (g) | | | | | |
| Voidage (%) | 55.6 | N/A | 49.0 | 50.8 | N/T |
| Increase in | 22 | N/A | 71 | 39 | 27 |
| mass on frying | | | | | |
| (%) | | | | | |

* Sunflower Lecithin was deoiled lecithin from Cargill Foods; N/A = not applicable because no product could be made; N/T = no measurement taken; RP = RuBisCo Protein.

It will be seen that, in spite of differences in volume, form and texture, doughnuts were formed when dried whole egg, dried egg white and Rubisco was present. No doughnut was formed from the egg free recipe (Trial 2, FIG. 5B).

It can be concluded that RuBisCo can serve as an egg replacer in cake doughnut.

### Example 4: Commercial Egg Replacers in Sponge Cake

There are several vegan ingredient blends that have claimed use in replacing egg-including the replacement of egg in bakery products. For reference purpose, we evaluated two such products: Orgran “No Egg, Egg Replacer for Baking” and Follow-your-Heart “Vegan Egg”. Ingredients listed are provided in Table 10

TABLE 10
Two examples of vegan egg replacers

| | Orgran, No Egg | Follow-you-Heart, Vegan Egg |
|---|---|---|
| Claims | For baking | For baking and scrambling |
| Ingredients | Potato Starch, Tapioca | Soymilk Powder (soybeans), |
| | Starch, Raising Agent | Modified Cellulose, Gellan Gum, |
| | (calcium carbonate), | Cellulose, Calcium Lactate, |
| | Acidity Regulator | Carrageenan, Black Salt (black |
| | (citric acid), Stabiliser | salt, herbs), Nutritional Yeast, |
| | (methylcellulose) | Natural Flavors, Beta-carotene |
| | | for color |
| Use | To replacer one whole | To replace one whole egg, 2 |
| Instructions | egg, use 3 g-6 g | tablespoons products plus ½ |
| for | product plus | cup iced water vigorously |
| baking | 30 ml water | whisked (estimated as being |
| | | equivalent to 10 g product per |
| | | 120 ml water) |

Using the method described in Example 1, Organ No Egg and Follow-your-Heart Vegan Egg were used according to the formulations of Table 11. The mass of whole liquid egg was replaced by the commercial egg replacer, that had been blended with water to fit as far as possible within usage recommendations to replace egg while keeping other ingredients at a constant level. For reference purposes, certain recipes from Example 1 were also included: whole liquid egg (Cake E), dried egg white (Cake G), egg-free with no replacer (Cake H), and egg-free with RuBisCo (RP #1; Cake A).

The recipes followed are shown in Table 11. Where egg white was replaced with Orgran, Follow your Heart at low level or wheat starch, cakes were too fragile to be removed from their tins properly-preventing any measurements to be made on these. Follow your Heart at the higher level gave a batter that was too thick to aerate during whipping and which then did not form into an acceptable sponge cake during baking. Sponge cakes with egg (Reference cakes E and G) and with RuBisCo (Cake A) made acceptable products. Properties were not measured in this instance—but were in line with their counterparts described in Table 3 and FIG. 2.

TABLE 11
Orgran No Egg preblended with water before being added to dry ingredients.

| | Cake E | | | Cake G | Cake K | Cake L | Cake H | |
|---|---|---|---|---|---|---|---|---|
| | Ref. | | | Ref. | Follow | Follow | Ref. | Cake A |
| | Whole | Cake I | Cake J | Dried | Your | Your | Egg Free | Egg |
| | liquid | Orgran | Orgran | white | Heart | Heart | (no | Free + |
| Ingredient | egg | Low | High | only | Low | High | replacer) | RP#1 |
| Cake wheat flour | 26.0 | 26.0 | 26.0 | 26.0 | 26.0 | 26.0 | 26.0 | 26.0 |
| Sugar (granulated) | 27.7 | 27.7 | 27.7 | 27.7 | 27.7 | 27.7 | 27.7 | 27.7 |
| Wheat starch | 1.6 | 1.6 | 1.6 | 6.2 | 1.6 | 1.6 | 8.3 | 6.2 |
| Sponge emulsifier | 2.6 | 2.6 | 2.6 | 2.6 | 2.6 | 2.6 | 2.6 | 2.6 |
| Sunflower Lecithin | | | | | | | | |
| SAPP 15 | 1.04 | 1.04 | 1.04 | 1.04 | 1.04 | 1.04 | 1.04 | 1.04 |
| Sodium bicarbonate | 0.76 | 0.76 | 0.76 | 0.76 | 0.76 | 0.76 | 0.76 | 0.76 |
| Salt | 0.10 | 0.10 | 0.10 | 0.10 | 0.10 | 0.10 | 0.10 | 0.10 |
| Potassium sorbate | 0.21 | 0.21 | 0.21 | 0.21 | 0.21 | 0.21 | 0.21 | 0.21 |
| Dried egg white | | | | 2.1 | | | | |
| Whole liquid egg | 40.0 | | | | | | | |
| Orgran No Egg | | 3.6 | 6.7 | | | | | |
| FYH Vegan Egg | | | | | 3.1 | 5.7 | | |
| RuBisCo Protein | | | | | | | | 2.1 |
| Water | | 36.4 | 33.3 | 33.3 | 36.9 | 34.3 | 33.3 | 33.3 |
| Total | 100 | 100 | 100 | 100 | 100 | 100 | 100 | 100 |
| Batter density (g/ml) | 0.51 | 0.44 | 0.46 | 0.44 | 0.61 | 0.95 | 0.41 | 0.41 |
| Removal of cake from | Yes | No | No | Yes | No | No | No | Yes |
| tin | | | | | | | | |

Follow Your Heart Vegan Egg preblended and whisked with ice-cold water before being added to dry ingredients; RP#1 = Rubsico sample at equal level to egg white protein of Ref B; RP#1.5 and RP#2 at 1.5x to egg white protein of Ref B; Sunflower Lecithin was de-oiled lecithin from Cargill Foods.

### Example 5: Commercial Egg Replacers in Creme Cake

Using the method of Example 2, Organ No Egg and Follow-your-Heart Vegan Egg were used following the design of Table 12. The mass of whole liquid egg was replaced by the commercial egg replacer, that had been blended with water to fit as far as possible within usage recommendations to replace egg while keeping other ingredients at a constant level. For reference purposes, certain recipes from Example 2 were also included: whole liquid egg (Cake F), dried egg white (Cake H), egg-free with no replacer (Cake I), and egg-free with RuBisCo (Cake A).

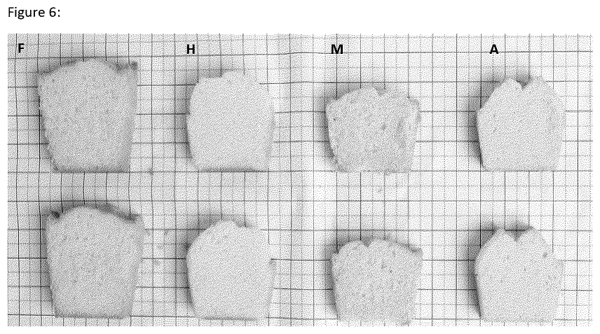

Results are shown also in Table 12 with images to the cakes provided in FIG. 6.

It will be seen that cakes J and K using Orgran were too fragile to be removed from their baking tin (being comparable then to Cake G made using wheat starch in place of egg white protein). The replacer “Follow-your-Heart” at higher level gave cake at least that could be removed from the baking tins (cake M), but both volume and textural firmness were lower than the counterpart using egg white (i.e. Cake H). This contrasted to cake made with RuBisCo: here volume was lower against Cake H, but there was gain in textural firmness (Cake A).

TABLE 12
Examples of commercial vegan egg replacers in creme cake

| | Cake F | | | Cake H | Cake L | Cake M | Cake I | |
|---|---|---|---|---|---|---|---|---|
| | Ref. | | | Ref. | Follow | Follow | Ref. | Cake A |
| | Whole | Cake J | Cake K | Dried | Your | Your | Egg Free | Egg |
| | liquid | Orgran | Orgran | white | Heart | Heart | (no egg | Free |
| Ingredient | egg | Low | High | only | Low | Height | replacer) | RP#1 |
| Cake wheat flour | 24.4 | 24.4 | 24.4 | 24.4 | 24.4 | 24.4 | 24.4 | 24.4 |
| Sugar (caster) | 24.4 | 24.4 | 24.4 | 24.4 | 24.4 | 24.4 | 24.4 | 24.4 |
| Wheat starch | | | | 4.4 | | | 5.9 | 4.4 |
| Distilled monoglyceride | 0.29 | 0.29 | 0.29 | 0.29 | 0.29 | 0.29 | 0.29 | 0.29 |
| SAPP 28 | 0.98 | 0.98 | 0.98 | 0.98 | 0.98 | 0.98 | 0.98 | 0.98 |
| Sodium bicarbonate | 0.68 | 0.68 | 0.68 | 0.68 | 0.68 | 0.68 | 0.68 | 0.68 |
| Salt | 0.24 | 0.24 | 0.24 | 0.24 | 0.24 | 0.24 | 0.24 | 0.24 |
| Dried egg white | | | | 1.47 | | | | |
| RuBisCo Protein | | | | | | | | 1.47 |
| Rapeseed Oil | 19.6 | 19.6 | 19.6 | 19.6 | 19.6 | 19.6 | 19.6 | 19.6 |
| Sunflower Lecithin | | | | | | | | |
| Whole liquid egg | 24.4 | | | | | | | |
| Orgran No Egg | | 2.25 | 4.1 | | | | | |
| FYH Vegan Egg | | | | | 1.9 | 3.5 | | |
| Water | 4.9 | 27.1 | 25.3 | 23.5 | 27.5 | 25.9 | 23.5 | 23.5 |
| Total | 100 | 100 | 100 | 100 | 100 | 100 | 100 | 100 |
| Batter density (g/ml) | 0.99 | 1.19 | 1.16 | 1.10 | 1.15 | 1.15 | 1.14 | 1.12 |
| Removal of cake from tin | Yes | No | No | Yes | No | Yes | No | Yes |
| Cake specific vol. (ml/g) | 2.6 | N/T | N/T | 1.7 | N/T | 1.8 | N/T | 1.6 |
| Crumb hardness (g) | 1009 | N/T | N/T | 2991 | N/T | 593 | N/T | 3406 |

1. A dough, batter or instant food mixture, comprising a non-denatured ribulose-1,5-bisphosphate carboxylase-oxygenase (RuBisCo) preparation, a starch, a sweetener, a lecithin and water.

2. The dough, batter or instant food mixture according to claim 1, wherein said RuBisCo preparation has a purity of at least 80% protein of the total dry mass of the RuBisCo preparation.

3. The dough, batter or instant food mixture according to claim 1, wherein said RuBisCo preparation comprises RuBisCo from duckweed, algae, beetroot, spinach, spinach beet, sea beet, Mangel beet, carrot, sugar beet, corn, soy or tobacco, most preferably a RuBisCo protein from duckweed.

4. The dough, batter or instant food mixture according to any of claim 1, wherein the lecithin comprises or essentially consists of one or more vegetable lecithin; or wherein the lecithin comprises at least one vegetable lecithin selected from the group consisting of soy lecithin, fava bean lecithin, garbanzo lecithin, sunflower lecithin, canola lecithin, rapeseed lecithin, corn lecithin, rice bran lecithin and cottonseed lecithin; or a combination thereof.

5. (canceled)

6. The dough, batter or instant food mixture according to claim 1, having a total lecithin content between about 0.1 wt. % and about 4 wt. % of lecithin, based on the total weight of batter, dough or instant food mixture.

7. The batter, dough or instant food preparation according to claim 1, comprising, based on the total weight of the batter or dough, between about 0.25 wt. % and about 5 wt. % of non-denatured RuBisCo preparation, or, based on the total weight of said instant food mixture, between about 0.5 wt. % and about 8 wt. % of the non-denatured RuBisCo preparation.

8. (canceled)

9. The dough, batter or instant food mixture according to claim 1, wherein the dough, batter or instant food mixture is a cake dough, a cake batter or instant cake mixture.

10. The dough, batter or instant food mixture according to claim 1, comprising a flour selected from wheat flour, spelt flour, corn flour, oat flour, barley flour, rye flour, sorghum flour, buckwheat flour, millet flour, triticale flour, amaranth flour, teff flour, rice flour, quinoa flour, tapioca flour, potato flour, chickpea flour, coconut flour, almond flour, cassava flour, arrowroot flour, or combinations thereof.

11. The batter according to claim 1, comprising, based on the total weight of the batter, 22-28 wt. % of wheat flour, 24-30 wt. % of sugar, 1.5-4 wt. % of the RuBisCo protein preparation and 30-40 wt. % of water, which batter is a sponge cake batter or comprising, based on the total weight of the batter, 18-24 wt. % of wheat flour, 22-28 wt. % of sugar, 1.2-4 wt. % of the RuBisCo protein preparation and 20-30 wt. % of water, 15-25 wt. % of fat, wherein the batter is a pound or crème cake batter.

12. (canceled)

13. The batter or dough according to claim 1, comprising, based on the total weight of the batter or dough, 35-45 wt. % of wheat flour, 17-21 wt. % of sugar, 0.5-2 wt. % of the RuBisCo protein preparation, 25-35 wt. % of water, 5-10 wt. % of fat.

14. The batter or dough according to any of claims 11-131, further comprising 0.5-2 wt. % of lecithin.

15. A heated food product comprising a heated dough or batter according to claim 1.

16. The heated food product according to claim 15, wherein the food product is a cake.

17. The heated food product according to claim 15, wherein said food product has a crumb hardness as determinable with a TA.XTplusC device from Stable Micro Systems Ltd that differs at most 20% from the crumb hardness of a comparable reference cake comprising dried egg white instead of a RuBisCo preparation, but of which the composition is otherwise the same.

18. The heated food product according to claim 15, having a RuBisCo preparation content between about 0.5 wt. % and about 8 wt. %, based on total ingredients other than water, and a lecithin content, between about 0.1 and about 4 wt. %, based on total ingredients other than water.

19. The dough, batter or instant food mixture according to claim 1, wherein the weight to weight ratio RuBisCo preparation content to lecithin content is in the range of about 1:5 to about 10:1.

20. The dough, batter or instant food mixture according to claim 1, comprising, based on the total weight of the dough, batter, instant food mixture, at most 20 wt. % of liquid whole egg, preferably chicken egg or wherein said dough, batter, instant food mixture is vegan, or a combination thereof.

21. (canceled)

22. A method for preparing a heated food product, comprising mixing a starch, a sweetener, a non-denatured RuBisCo preparation and water to form a batter or a dough; or mixing an instant food mixture, comprising a starch, a sweetener, a non-denatured RuBisCo preparation with water to form a batter or a dough; heating said batter or dough to a temperature above the denaturing temperature of said non-denatured RuBisCo to obtain the heated food product.

23. The method according to claim 22, wherein an inner part of said batter or dough is heated to a temperature above 90° C.

24. The method according to claim 22, wherein said batter or dough is baked, cooked, steamed or fried.

25. (canceled)

26. (canceled)

27. (canceled)

28. (canceled)

29. (canceled)
